(19) United States
(12) Patent Application Publication
(10) Pub. No.: US 2007/0208279 A1
(43) Pub. Date: Sep. 6, 2007

(54) Motor Function Test System

(75) Inventors: Lorenzo Panella, Milano (IT); Angelo Buizza, Pavia (IT); Remo Lombardi, Pavia (IT); Giorgio Beltrami, Pavia (IT); Roberto Gandolfi, Pavia (IT)

Correspondence Address: MERCHANT & GOULD PC, P.O. BOX 2903, MINNEAPOLIS, MN, 55402-0903, US

(73) Assignee: HUMANITAS MIRASOLE S.p.A., Milano (IT)

(21) Appl. No.: 10/576,962

(22) PCT Filed: Oct. 24, 2003
(86) PCT No.: PCT/IT03/00660

§ 371 Date: Jan. 12, 2007

(51) Int. Cl. A61B 5/103

(52) U.S. Cl. 600/595; 128/920

(57) ABSTRACT

A motor function test system as well as to a method of acquisition and collection of the signals are processed into the corresponding parameters for a motor function test. In particular, the system and method relate to the quantification of the performance of a subject during the evaluation of balance as defined by the Tinetti test.

The present finding relates to a motor function test system as well as to a method of acquisition and collection of signals and their processing into the corresponding parameters for a motor function test. In particular, said system and method relate to the quantification of the performance of a subject during the evaluation of balance as defined by the Tinetti test.

The Tinetti test is widely used for evaluating the control of posture and the motor capacity of elderly or disabled people. This test is based on a series of simple movements from daily life, which must be performed by the subject in a defined sequence. The performance of the subject is evaluated by the physician or by the physiotherapist who expresses their judgement through a numerical score based on a predefined reference scale.

From that already described, the outcome of the Tinetti test is largely subjective precisely because it relies on the evaluation criteria of the examiner. It is therefore apparent that the test may be distorted or however altered by said subjective nature. Only an objective test can ensure homogeneous evaluation criteria and, hence, the possibility of comparing different evaluations, i.e. both evaluations performed by different staff on the same patient and evaluations performed by the same staff on different patients.

The problem at the heart of the present invention is therefore to devise a motor function test system which allows the achievement of objective evaluations.

Such a problem is resolved by a motor function test system as covered by the attached claims.

A first subject of the present invention is therefore that of providing a motor function test system which exploits the measurement of biomedical parameters and/or functions correlated with the movement of the subject and/or with their posture control during the Tinetti test.

A second subject of the present invention is that of providing a method for the acquisition and collection of signals and their processing into the corresponding parameters for a motor function test, as provided by said motor function test system.

Further characteristics and advantages of the aforementioned system and method shall become evident from the following description of an embodiment of the invention, given for non-limiting exemplification, with reference to the attached figures wherein:

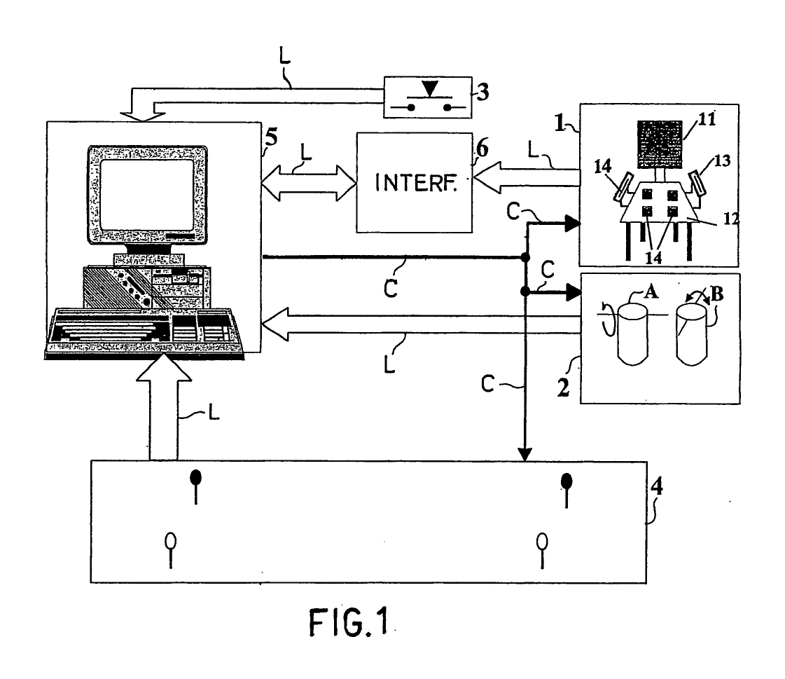

FIG. 1 represents a functional block diagram of the system of the invention;

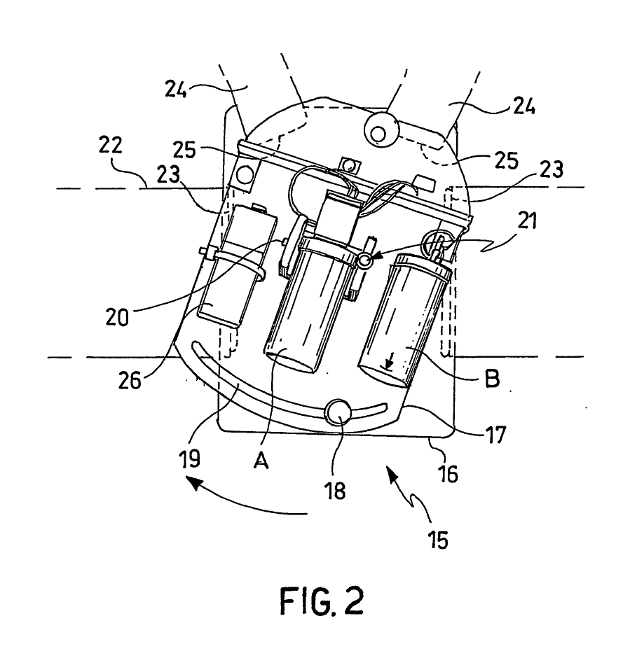

FIG. 2 represents, as a useful example for the description, but neither binding nor limiting, a pair of inclinometers of the system of FIG. 1 to be applied to the chest of a subject.

The motor function test system as represented schematically in FIG. 1 comprises various functional units operatively connected to one another. In particular, said system comprises a chair 1 for a motor function test, means 2 suitable for detecting inclinations of the torso of a person subjected to said motor function test, a button 3 (marker) with related power supply, at least one pair of optical detectors 4, an electronic data processor 5, an interface 6. In FIG. 1, analogical and/or digital signal lines L and power cables C are also represented.

The chair 1 is of the type with armrests, endowed with a backrest 11 (optional), a seat 12 and two armrests 13. The backrest 11 and the seat 12 are substantially rigid. Furthermore, the seat 12 and the armrests 13 are each fitted with at least one, thin, conventional-type pressure sensor 14, on a flexible support. Thin upholstery can be envisaged in such a manner as to cover and protect said at least one pressure sensor 14 and, at the same time, make the seat 1 more comfortable. Each pressure sensor 14 is of such as to produce an electrical signal correlated with the pressure exerted on it by the subject subjected to the test. Preferably, said pressure sensor 14 is represented by a thin resistive sensor, made from a sensitive film placed between two flexible polymeric sheets. In general, the following nominal characteristics may be considered adequate: a full scale of 500 kPa, a maximum applicable pressure of 2500 kPa, a response time of less than 20 ms, a sensitive area and shape such as to conform them to the dimensions of the seat and the armrests.

In particular, in the exemplificative embodiment as per point [0008], on the seat 12 said at least one sensor is represented by four square sensors 14, of the type Interlink Electronics Europe FSR154, arranged in a 2×2 matrix, whilst on each of the armrests 13 there is a rectangular sensor of elongates shape, of the type Interlink Electronics Europe FSR648AS, with the long side aligned with the axis of the armrest itself. Said sensors 14 have the following nominal characteristics:

range of measurement: from 69 Pa to 689 kPa;

maximum pressure: ≅3400 kPa;

response time≅2 ms;

typical load free resistance: 0.4 MΩ/cm², which diminishes progressively with increasing load;

sensitive area approx. 14 cm².

In this exemplificative embodiment, furthermore, said sensors 14 are connected to the electronic data processor 5 through cables and the relative connectors, entirely conventional and therefore not shown, suitable f or the transmission of the signals produced by the sensors and for the supply of power for the sensor themselves. Advantageously, in addition, the cable connectors are positioned below the seat 12 together with a capacitor, itself also conventional and not shown, having the task of filtering the voltage of the DC power source for the sensors. In addition, one or more integrated circuits may be present, for a total number of functional amplifiers (four in said exemplificative embodiment) sufficient to make suitable low-pass anti-aliasing filters, considering the subsequent analogue-digital conversion of the pressure signals. Furthermore, the power supply of the sensors 14 may be realized by batteries placed for example under the seat 12 and/or the armrests 13.

The means 2 suitable for detecting inclinations of the torso of a person may preferably comprise a pair 2 of inclinometers, or possibly a mono- or bi-axial accelerometer, to be applied to the torso.

Said pair 2 of inclinometers is represented by a first inclinometer A appointed to measure the inclination of the torso in the anterior-posterior plane and a second inclinometer B appointed to measure the inclination of the torso in the mediolateral plane. In general, said pair of inclinometers should have a full scale of at least ±25°. In the event that accelerometers are used, the procedure is analogous.

Preferably, said pair of inclinometers is represented by two mechanical-electrical inclinometers of the type Midori Precision PMP-S30TX, shown in FIG. 2, with the following nominal characteristics:

supply voltage (V₀): from 4.5 to 8 V DC;

full scale: ±30°;

corresponding output voltage range: 2.5 V (offset)±1.1 V, with supply V₀=8 V;

linearity: ±1% FS;

sensitivity (including hystereses) better than 0.03°;

output sensitivity: 0.00225 V₀±2% V/° (V₀ in 20 Volts);

response time≈0.2 s;

dimensions: diameter=20 mm, height=40 mm;

weight: 35 grams;

buffering in 200CS siliconised oil.

In the event that the full scale of the pair of inclinometers 2 is less than ±45°, it is advisable that the pair of inclinometers 2 be mounted on a suitable support 15 such as for example a polycarbonate support as schematically represented in FIG. 2. In general, the support 15 must be constructed in such a manner that the corresponding planes of maximum sensitivity of the inclinometers A and B are perpendicular to one another. Indeed, when the pair 2 of inclinometers A and B is fixed onto the chest of an individual, as will be better described in the following, the aforementioned two planes must be substantially parallel to the medial and frontal planes respectively of said individual. The support in question must allow the orientation of the planes of maximum sensitivity of each inclinometer according to the direction of gravity, when the inclinometers are applied to the chest of the patient and they assume the normal erect posture. Such alignments are carried out on each subject prior to the beginning of the test (see below) and has the purpose of allowing the best exploitation of the dynamics of the sensor and of limiting the risk of saturation of the measurement during the execution of the movements which require wider inclinations of the torso. This requirement does not arise if the measurement range is sufficiently wide, i.e., beyond ±45°, as already mentioned.

For this reason, the support 15 may be constituted, as in the exemplificative embodiment already repeatedly mentioned, by two parallel plates: one inner 16 and one outer 17 connected to one another in such a manner as to rotate with respect to one another around an axis perpendicular to their planes. The inner plate 16, when the support is mounted on a subject, is placed in contact with the body of said subject, whilst the plate 17 is external with respect to said body.

In particular, the inner 16 and outer 17 plates have, in the said exemplificative embodiment, substantially rectangular shape and, as represented in FIG. 2, said connection is made in the proximity of one of the sides of said plates 16 and 17. The means of connection comprise a pivot 18 fixed to the inner plate 16 and a thin window 19 which extends in a circular arch shape formed in the outer plate 17. The window 19 engages said pivot 18 in such a manner as to allow the rotation of the outer plate 17 over the inner plate 16. This rotation, once a subject has been made to wear the inclinometers A and B using the devices (elasticized strap 22 and braces 24) as per the following point, allow the alignment of the plane of maximum sensitivity of the inclinometer B with respect to the direction of gravity. The alignment of the corresponding plane of maximum sensitivity of the inclinometer A takes place by leaving the inclinometer itself free to oscillate around an axis 20 perpendicular to the axis of the inclinometer A and then fixing this latter, in the position thus assumed, using a locking screw 21.

The inner plate 16 is then movably mounted on an elasticized strap 22 using two buttonholes 23 formed on two of its opposing sides. In addition, said inner plate 16 is also engaged by two braces 24 through two corresponding buttonholes 25. The strap 22 and the braces 24, as will be described later, have the role of allowing the wearing of the pair of inclinometers 2 at the level of the torso on a person.

The outer plate 17 constitutes the actual support for the inclinometers A and B. Indeed, as represented in FIG. 2, on its surface turned in the opposite direction with respect to the inner plate 16, are mounted an inclinometer A, through the axis 20 and related holding screws 21 as described above, an inclinometer B fixed rigidly to said outer plate 17, a capacitor (not shown) for the filtering of the DC supply voltage and one or more integrated circuits (IC, not shown) containing a suitable number of operational amplifiers (four in said exemplificative embodiment) to be used as output buffers and as anti-aliasing filters in view of the subsequent analogue-digital conversion of the signals from the inclinometer.

Preferably, a battery 26 of suitable voltage and charge may also be mounted on the outer plate 17 as a potential independent energy source for the inclinometer, equipped with the necessary circuitry for the supply and with a switch (not shown) in order to be able to select as an energy source, the aforementioned battery, or the external power supply deriving from the electronic data processor 5 or from other sources.

The button 3 may be entirely conventionally hand or pedal operated, and is supplied with low voltage power, from the data processor 5 or from an independent battery (for example type AA penlight). It is operated by the physiatrist or physiotherapist who administers the motor function test and may be used as a marker to indicate the start and possibly the end of the various stages of which the motor function test is composed. When pressed, it produces a constant voltage signal, which will be acquired by the electronic data processor 5, together with the signals produced by the other components of the system.

It should be noted that the button 3 may be substituted by any other means which allows the marking of the beginning and the end of each stage of the test, such as, for example, a keyboard or mouse command from the electronic data processor 5 itself. As a consequence, the various stages of the test may be distinguished from one another without any ambiguity, with the aim of correct interpretation and analyses of the signals which are obtained by the electronic data processor 5.

In accordance with one preferred embodiment, the motor function test system may comprise, alternatively or in addition to the previous embodiment, at least one pair 4 of optical detectors positioned along a route which the subject must encounter. In particular, said optical detectors are represented by photoemitter-photoreceiver combinations, with or without the insertion of a reflector in the optical path, or by other conventional devices able to detect the passing of a subject. Preferably, said detectors may be positioned at the beginning and end of the route respectively in such a manner as to record the time of departure and that of arrival.

The electronic data processor 5 may be for example constituted by an entirely conventional desktop (PC) or portable personal computer (including a central processing unit CPU and related RAM and ROM memory) and endowed with likewise conventional peripherals (HDD, FDD, CD-ROM drive, possible CD-ROM writer, graphics printer) and user interfaces (in particular keyboard and mouse or equivalent devices).

The personal computer 5 is endowed with a data acquisition module (not shown), comprising a analogue-digital conversion card endowed with at least 12 bit A/D input ports, in sufficient numbers to acquire all the signals of interest. According to the present example embodiment, at least the following signals are of interest: a signal for each of the two inclinometers 2, plus possibly a signal for each of the two pairs of optical detectors 4, plus a signal for the button 3, plus a number of additional signals, equal to the number of pressure sensors 14 actually used. Preferably, the same data acquisition module will be endowed with digital output ports to be used for possibly supplying power to the inclinometers 2, the button 3 (if not supplied independently) and the pressure sensors 14, whilst the optical detectors 4 will require, in general, an independent power supply, from a battery or the mains. Analogously, the same data acquisition module could be endowed with digital input ports, for example of the type TTL, to be used for other possible commands which should be deemed appropriate or necessary for the good operation of the entire system. The said input and output ports are not represented in the figure in as much as such devices are widely known in the sector. This data acquisition module, thanks to its analogue-digital conversion function, in particular allows the sampling the signals originating from the pressure sensors 14, from the inclinometers A and B, from the button 3 and from the optical detectors 4. Such sampled signals represent, for example in the case of the inclinometers A and B, the results of the related inclination measurements of the torso during the movements performed by the subject in accordance with the Tinetti test. The sequence of their values, appropriately expressed in sexagesimal degrees or in other angular measurement units, represent the variation over time of the angle of inclination of the torso with respect to the vertical axis, in the medial and frontal planes, during said test. Analogously, the electrical signals produced by the pressure sensors 14 measure as a function over time the values of the pressure exerted by the subject on the seat 12 and on the armrests 13. The signals produced by the button 3 are represented by impulses which indicate the exact moments of the beginning and end of each of the different stages of which the motor function test is composed. Analogously, the signals emitted by the optical detectors 4 are represented by impulses, produced in the exact moments in which the subject crosses the line of detection of the detectors themselves, at the beginning and at the end of the envisaged route or at the other points where optical sensors have possibly been placed. All the aforementioned sampled signals, together with the identifying data of the subject and of the test believed necessary, may be usefully organised into a database set up on the personal computer 5 itself or on another computer or stored on whichever other computer media and organised into whichever other form may be deemed relevant and appropriate.

The sampled signals as per the previous point will be processed according to the criteria and methods illustrated later, with the aim of obtaining numerical parameters, suitable for objectively characterising the performance of the subject during the execution of the test. All the parameters thus obtained will be then compared with pre-established reference parameters. Through this comparison, the physician will have objective information available for an accurate and completely objective evaluation of the posture control and the balance of the patient, as resulting from the Tinetti test.

A program for the processing of the sample signals acquired as described above and for the calculation of said numerical parameters is loaded within the RAM memory of the above mentioned personal computer 5 or of another personal computer. The same program may be made available to the above mentioned personal computer 5 or other personal computer through floppy disk, CD-ROM or other method known within the sector such as for example methods which envisage transmission through a telecommunications network.

The central processing unit of the personal computer 5 or other personal computer, is of such as to run the instructions of the program processing said sample signals.

Furthermore, said system may comprise an interface 6 used for enabling/disabling the acquisition of the pressure signals or of the signals from the other sensors, for example by means of BJT switches controlled through the digital output ports. Preferably, said interface 6 is located externally with respect to the personal computer. Alternatively, one might resort to the selection via software of the signals to be acquired.

In accordance with an additional variant embodiment, the motor function test system of the present invention envisages, alternatively or in addition to the preceding embodiments, that the transmission of the signals produced by the inclinometric sensors 2 to the personal computer 5 be carried out through wireless technology, with the aim of ensuring the complete freedom of movement of the subject during the execution of the test, and to avoid any possible obstructions caused by the connecting cables. In this case, the inclinometers A and B are connected to a transmission module, for example, mono-channel or dual-channel (not shown), intended to carry out the processing of the signals produced by the inclinometric sensors 2 themselves (possible sampling and A/D conversion, possible manipulation, modulation, amplification and the rest) in order to make them suitable for irradiation through free space, practicable through an appropriate antenna. Consequently, the acquisition module is equipped with an additional antenna and a receiver module (not shown) in order to carry out the necessary processing (demodulation, demultiplexing and the rest) of the irradiated signals received. For example, it is possible to use radiofrequency systems of the type known within the sector. One particular system may be represented, for example, by an embodiment according to the Bluetooth 1.1 international standard, which has adequate characteristics for the transmission of said signals and the components of which are available commercially.

It is to be noted that the above mentioned wireless technology may also be applied to the pressure sensors 14 and to the optical detectors 4.

A second subject of the present invention is that of providing a method for detection, collection and processing of parameters for a motor function test comprising the following stages in sequence:

a) making available a system for a motor function test such as that previously described;

b) applying to a subject for testing means 2 suitable for detecting inclinations of the torso of said subject;

c) detecting the pre-established movements of that subject by means of said means 2 and possibly at least one pressure sensor 14 and possible optical detectors 4;

d) transmitting signals corresponding to said detection performed in stage c) to the electronic data processor 5;

e) collecting and processing said signals originating from said at least one pressure sensor 14 and/or from said means 2 and/or from said optical detectors 4 in such a manner as to obtain parameters representative of the ambulatory and/or the posture control of said subject.

In particular, stage c) comprises a series of pre-established movements recordable by the sensors, such as those previously described. Said sensors are able to send signals to the personal computer 5 which will acquire them and will process them in such a manner as to calculate numerical parameters, useful for evaluating the motor capacity of a subject.

The movements may be established each time according to the subject type and/or his motor problems. For example, the subject may be initially placed sitting on the chair 1 with the pair 2 of inclinometers worn thanks to the above described strap 22 and braces 24. In this position, the inclinometers A and B are aligned with the vertical axis, which will serve as a reference for the inclination of the torso. Afterwards, the subject is asked to make the following movements in accordance with the Tinetti test:

movement 1: the subject is asked to maintain the seated position with the hands resting on the knees/thighs;

movement 2: the subject is asked to rise from the seated position in a natural manner and without the aid of the hands;

movement 3: the subject is asked to remain standing, after having risen from the chair, in an erect position for approximately 20 seconds with the eyes open (the first 4 seconds are considered as “immediate standing”, the remaining as “prolonged standing”);

movement 4: the subject is asked to maintain the erect position for approximately 15 seconds with the eyes closed;

movement 5: the subject is asked to make a complete 360° turn around themselves on the spot;

movement 6: the subject is asked to resist three light pushes applied by the physician at the level of the sternum;

movement 7: the subject is asked to perform a direct walking movement between the pair of photocells 4;

movement 8; the subject is asked to sit down again on the chair 1, which the operator has brought closer in the meantime.

Stage c) comprises the detection of the movements of the subject during the test through means of detection which may comprise, for example the above mentioned pressure sensors 14, inclinometers 2 and optical detectors 4. These means of detection produce electrical signals which may be transmitted to the personal computer 5 through cables or, in accordance with the above mentioned preferred embodiment, wirelessly.

In particular, the pressure sensors 14 placed on the seat 12 serve to monitor the presence of the subject, or rather whether they are seated or not, and, in the case they are seated, if their posture is symmetrical or unbalanced towards the right or towards the left. Whilst the pressure sensors 14 on the armrests 13 serve to verify whether that subject rests on them during the standing up and/or sitting down movements, and in the affirmative case, whether they rest equally on both armrests or not.

The optical sensors 4 record the moment of departure and that of the arrival of the subject along the distance which separates them.

Stage e) comprises the automatic calculation, performed by the computer 5 or other data processor, of a collection of numerical parameters able to quantify the performance of the subject during the execution of the test and, in particular, allowing the classification of such performance as normal or altered. To that end, a program for the processing of the signals acquired and for the calculation of said numerical parameters, as already mentioned in point [0026] is loaded within the RAM memory of the above mentioned personal computer 5, or other personal computer.

Preferably, the above mentioned numerical parameters will be morphological in nature (such as: measurements of breadth, duration, speed, etc.) and different for the various stages of which the test is composed. They may be further processed and combined, by using the techniques known within the sector, preferably with the aim of obtaining a single performance index, on the basis of which, and for comparison with suitable normal reference values, the physician may express a judgement of normality or of abnormality with reference to the performance of the subject. Alternatively, other forms of parameterisation and/or other performance indices which will show themselves to be useful and appropriate for the evaluation of the performance of the subject may be adopted.

The method in accordance with the present invention may envisage the use of the previously described system comprising the pressure sensors 14 placed on the armrests 13 of the chair 1. Thanks to them, it is possible to evaluate whether the subject uses the armrests 13 for standing up or sitting down and measure the symmetry of any resting and its amount, and, hence, its importance regarding the execution of the task. In this manner, the method is advantageously enriched with an objective data point, useful with regard to better characterisation of performance. The presence of the armrests and the possibility of using them, in addition, increases the degree of composure and safety of the patient and contributes towards reducing the risk of falling.

Alternatively or in combination, the method of the invention may also comprise the use of the above described optical detectors 4 arranged along a route which the subject must encounter. The predisposition of said optical detectors 4 advantageously allows the provision of an additional data point regarding the moments of departure and arrival of the subject along the route between said detectors and hence of measuring the journey time with greater accuracy, which, in turn, will depend on good or bad ambulation. It follows that an additional indicator of the possibility of falling may be observed and taken into consideration.

Furthermore, still alternatively or in combination, the above mentioned method may envisage a wireless data transmission system such as that previously described. It is apparent that such a system is extremely advantageous since it allows full freedom of movement to the subject who must perform the Tinetti test and hence the most natural test conditions possible, something which would not be equally guaranteed by cable such as it is.

Furthermore, detection parameters are introduced which without doubt improve the objectivisation of the test result and the evaluation of the risk of falling of a subject subjected to the test.

Finally, the execution conditions of the test are once again notably improved in favour of an optimal result such as was not possible with the systems of the known art.

1. A motor function test system comprising a chair for a motor function test comprising a seat endowed with at least one pressure sensor and armrests each endowed with at least one pressure sensor, means suitable for detecting inclinations of the torso of a subject, an electronic data processor to receive signals emitted by said at least one pressure sensor and from said means when stimulated, and to collect said signals and process the corresponding descriptive parameters.

2. The motor function test system according to claim 1, further comprising at least one pair of optical detectors placed at the beginning and at the end of an established route which said subject must encounter.

3. The motor function test system according to claim 1, wherein the transmission of the signals from said pressure sensors, from said means and from said pair of optical detectors to said electronic data processor is carried out using wireless technology.

4. The motor function test system comprising a chair for a motor function test endowed with at least one pressure sensor positioned on the seat, means suited to detecting inclinations of the torso of a subject, at least one pair of optical detectors placed at the beginning and end of an established route which a subject must encounter, an electronic data processor to receive the signals emitted by said at least one pressure sensor, by said means and by said pair of optical detectors when stimulated, and to collect said signals and process the corresponding descriptive parameters.

5. The motor function test system according to claim 4, wherein the transmission of the signals from said pressure sensors, from said means and from said pair of optical detectors and said electronic data processor is carried out using wireless technology.

6. The motor function test system comprising a chair for a motor function test endowed with at least one pressure sensor positioned on the seat, means suited to detecting inclinations of the torso of a subject, an electronic data processor to receive the signals emitted from said at least one pressure sensor and from said means when stimulated, and to collect said signals and process the corresponding parameters, wherein the transmission of the signals to said electronic data processor is carried out using wireless technology.

7. The motor function test system according to claim 1, further comprising an interface for enabling/disabling the acquisition of said signals towards said electronic data processor.

8. The motor function test system according to claim 1, wherein said at least one pressure sensor is mounted onto a flexible support.

9. The motor function test system according to claim 8, wherein said at least one pressure sensor is a thin resistive sensor made with a sensitive film placed between two flexible polymeric sheets, wherein said at least one pressure sensor is of the type Interlink Electronics Europe FSR154 on the seat, is and wherein said at least one pressure sensor of the type Interlink Electronics Europe FSR648AS on the armrests.

10. The motor function test system according to claim 1, wherein said means suitable for detecting inclination of the torso of a subject comprise a pair of inclinometers.

11. The motor function test system according to claim 4, wherein said means suitable for detecting inclination of the torso of a subject comprise a pair of inclinometers.

12. The motor function test system according to claim 6, wherein said means suitable for detecting inclination of the torso of a subject comprise a pair of inclinometers.

13. The motor function test system according to claim 10, wherein said pair of inclinometers comprise a first inclinometer appointed to measure the inclinations of the torso of a subject in the anterior-posterior plane and a second inclinometer appointed to measure the inclinations of the torso of said subject in the mediolateral plane.

14. The motor function test system according to claim 11, wherein said pair of inclinometers comprise a first inclinometer appointed to measure the inclinations of the torso of a subject in the anterior-posterior plane and a second inclinometer appointed to measure the inclinations of the torso of said subject in the mediolateral plane.

15. The motor function test system according to claim 12, wherein said pair of inclinometers comprise a first inclinometer appointed to measure the inclinations of the torso of a subject in the anterior-posterior plane and a second inclinometer appointed to measure the inclinations of the torso of said subject in the mediolateral plane.

16. The motor function test system according to claim 10, wherein said pair of inclinometers are of the type Midori Precision PMP-S30TX.

17. The motor function test system according to claim 10, wherein said pair of inclinometers are mounted onto a support constructed in such a manner as to allow the orientation of the respective planes of maximum sensitivity of said inclinometers perpendicularly to one another.

18. The motor function test system according to claim 17, wherein said support comprises an inner plate and a parallel outer plate connected to one another in such a manner as to rotate one with respect to the other around an axis perpendicular to their plane.

19. The motor function test system according to claim 18, wherein said inner plate is movably mounted onto an elasticized strap through a buttonhole and is engaged with two braces through two corresponding buttonholes so that a subject may wear said pair of inclinometers.

20. The motor function test system according to claim 2, wherein said at least one pair of optical detectors are represented by two pairs of photocells or two pairs of photocell-reflectors or similar devices suitable for detecting the passage of a subject through them.

21. The motor function test system according to claim 7, wherein said interface enables/disables the acquisition of the electrical signals originating from said pressure sensors and/or pair of inclinometers and/or pair of optical detectors.

22. The motor function test system according to claim 3, wherein said transmission of the electronic signals uses radiofrequency systems, in particular carried out according to the Bluetooth 1.1 international standard or the like.

23. The motor function test system according to claim 1, further comprising a button connected to said electronic data processor to indicate the beginning and possibly the end of the various stages of which the motor function test is composed.

24. A method for the acquisition and collection of signals and their processing into the corresponding parameters for a motor function test comprising the following stages in sequence:
a) providing a motor function test system according to claim 1;
b) applying to a subject to be tested the means suitable for detecting inclinations of the torso of said subject;
c) detecting the pre-established movements of such subject by said means and possibly the at least one pressure sensor and possibly the optical detectors;
d) transmitting the signals corresponding to said detection achieved in stage c) to the electronic data processor;
e) collecting and processing said signals originating from said at least one pressure sensor and/or from said means and/or from said optical detectors in such a manner as to obtain parameters representative of the degree of ambulation and or posture of said subject.

25. The method according to claim 24, wherein said detection stage c) is achieved by detecting variations in pressure and/or inclination and/or the passing respectively between said pressure sensors, said means and said at least one pair of optical sensors.

26. The method according to claim 24, wherein said transmission stage takes place using cable or wireless technologies.

27. The method according to claim 24, wherein said stage of collection and processing of the signals originating from said pressure sensors and/or from said means and/or from said at least one pair of optical sensors by said electronic data processor comprises the transformation of the signals into the digital data from which said parameters are obtained.

28. The method according to claim 27, wherein the aforementioned parameters are numerical morphological parameters which may be further processed and combined with the aim of obtaining a single performance index.
